(19) United States
(12) Patent Application Publication
(10) Pub. No.: US 2004/0001881 A1
(43) Pub. Date: Jan. 1, 2004

(54) Transdermal therapeutic system for delivering lerisetron

(76) Inventors: Thorsten Selzer, Neuwied (DE); Petra Botzem, Andernach (DE); Gerd Hoffmann, Neuwied (DE); Heinz Kindel, Ehlscheid (DE)

Correspondence Address: BIRCH STEWART KOLASCH & BIRCH, PO BOX 747, FALLS CHURCH, VA, 22040-0747, US

(21) Appl. No.: 10/240,105

(22) PCT Filed: Mar. 17, 2001
(86) PCT No.: PCT/EP01/03089

(30) Foreign Application Priority Data
Mar. 30, 2000 (DE) 100 15 783.1

(51) Int. Cl. A61K 31/4178; A61K 31/716; A61K 9/70

(52) U.S. Cl. 424/449; 514/381; 514/57

(57) ABSTRACT

A lerisetron pharmaceutical preparation in the form of a transdermal therapeutic system (TTS) comprises a backing layer, connected to it an at least single-layer pressure-sensitively adhesive, lerisetron active substance reservoir based on silicone pressure-sensitive adhesive(s), and a removable protective layer.

The present invention relates to pharmaceutical preparations for administering the active substance lerisetron to the skin. It further relates to the use of such preparations for the transdermal administration of this active substance to patients for the prevention and therapy of nausea and vomiting.

The active substance lerisetron belongs to the class of the selective 5-HT3 receptor antagonists. It is generally suitable for treating nausea and vomiting. In particular, this active substance can be used to prevent or suppress the vomiting or the nausea induced by radiation therapy or chemotherapy.

When this active substance is administered orally, in particular in the course of a chemotherapy or radiation therapy, problems may occur, such as gastrointestinal intolerance, low enteral absorption, and rapid first-pass metabolization in the liver, for example. The last-mentioned effect in particular may necessitate more frequent administration.

Circumventing the gastrointestinal tract and thus the first-pass effect as well is possible in principle by transdermal administration of the active substance in question, using, for example, transdermal therapeutic systems (TTS). These are administration forms which are applied to the skin and which deliver the active substance present to the skin. In general, TTS are able to raise the therapeutic value of a pharmaceutical preparation by ensuring constant delivery of the pharmaceutical to the blood compartment over a prolonged period of time.

TTS of this kind typically have a composition comprising a medicament-impermeable backing layer, a medicament-containing reservoir layer, an optional control membrane, and also a pressure-sensitive adhesive layer for attachment to the skin, it being possible for the latter to be identical to the medicament-containing reservoir layer. The medicament-containing layer may also comprise further ingredients, examples being plasticizers, tackifiers, solubilizers, stabilizers, fillers, excipients and permeation enhancers. The pharmaceutically unobjectionable substances suitable for this purpose are known in principle to the person skilled in the art.

Although TTS as administration forms are known in principle, the formulation of a specific active substance, e.g. lerisetron, as a TTS represents a certain challenge, and a variety of problems may occur. For instance, in order to be suitable for therapeutic use, a TTS must permit a sufficiently high flux of active substance through the skin. Furthermore, it must possess good stability and in particular must not be subject to any changes during storage. The selection of appropriate polymers for the active substance reservoir may prove to be difficult, since these polymers must be compatible with the active substance in question. Furthermore, it must be possible to produce the TTS cost-effectively.

It was therefore an object of the present invention to provide a transdermal administration form for the active substance lerisetron that permits a sufficiently high active substance flux in vivo and can be produced cost-effectively by means of common production processes.

In accordance with the invention, this object is achieved by a lerisetron-containing pharmaceutical preparation in th form of a transdermal therapeutic system (TTS) according to claim 1, which comprises an at least single-layer, pressure-sensitively adhesive, lerisetron active substance reservoir based on silicone pressure-sensitive adhesive(s). These pressure-sensitive adhesives, and further polymers if desired, form the polymer matrix of the active substance reservoir. By silicone pressure-sensitive adhesives are meant pressure-sensitive adhesives based on a polydimethylsiloxane structure.

The active substance reservoir is connected to an active-substance-impermeable protective layer. Furthermore, the pharmaceutical preparation of the invention in the form of a TTS has a removable protective layer which is removed from the adhesive layer before the pressure-sensitively adhesive TTS is applied to the skin.

It has been found that the abovementioned formulation based on silicone pressure-sensitive adhesive(s) is particularly advantageous since lerisetron possesses on the one hand a high permeability in the silicone matrix of the active substance reservoir but on the other hand a low affinity for this matrix. Moreover, silicone pressure-sensitive adhesives possess high compatibility with the active substance lerisetron. A further advantage is that the adhesion properties remain constant even on fluctuations in temperature and humidity.

In accordance with the invention, lerisetron pharmaceutical preparations in the form of transdermal therapeutic systems according to claim 2 may also have an at least single-layer pressure-sensitively adhesive active substance reservoir which has been constructed on the basis of polymers selected from the group consisting of polyisobutylen s, polyterpenes, ethylene-vinyl acetate copolymers, synthetic rubb rs and hot-melt adhesives.

Furthermore, the leris tron active substance reservoir of the TTS of the invention may also have been constructed from a mixture of at least two polymers, it being possible for these polymers to be selected from the group consisting of silicone pressure-sensitive adhesives, polyisobutylenes, polyterpenes, ethylene-vinyl acetate copolymers, synthetic rubbers and hot-melt adhesives.

In principle, the active substance reservoir is constructed from an at least single-layer polymer matrix which comprises the active substance lerisetron and, if desired, the additional ingredients specified later on below.

In accordance with one particular embodiment, at least one polymer matrix layer of the active substance reservoir comprises polymer constituents from the group of the substituted celluloses, preferably from the group of the methyl celluloses or ethyl celluloses.

In principle, the active substance lerisetron may be present in molecularly disperse form or in solution in the active substance reservoir; also possible, however, is a formulation in which the active substance is present in coarsely disperse form, in colloidal form, or as a suspension.

It is preferred to aim for a concentration of active substance in the active substance layer(s) of the active substance reservoir that is as high as possible, in order to achieve a high rate of release (active substance flux). Where possible, the concentration of lerisetron should reach the saturation solubility; the active substance layers may also be supersaturated with active substance, in which case the saturation solubility is exceeded. However, it should be borne in mind that, with excessive concentrations of active substance, the physical stability of the active substance in the active substance reservoir may be adversely affected. In the case of the TTS of the invention, therefore, the aim is for active substance concentrations in the range from 0.1 to 30% by weight, particular preference being given to active substance concentrations in the range between 1 and 10% by weight; the concentration figures are based on the overall mass of the active substance layers.

If the active substance is to be present in dissolved form in the active substance reservoir, it is advantageous if the formulation of the polymer matrix of the active substance reservoir includes a solubilizer. Examples of such solubilizers that may be mentioned are the following: 1,2-propanediol, tetrahydrofurfuryl alcohol, Transcutol, butanediol, glycerol, PEG 400, diethyltoluamide, monoisopropylideneglycerol, a particularly preferred solubilizer being 1,2-propanediol. It has been found to be advantageous if the proportion of the solubilizer, based on the total TTS, is between 1 and 50% by weight, preferably between 5 and 35% by weight.

In certain circumstances, the incorporation of a solubilizer into the polymer matrix may produce a two-phase system, and/or the active substance lerisetron may be present as a dispersion. In these cases in particular it is advantageous to admix an emulsifier to the active substance polymer matrix.

In this case, the emulsifier is usually incorporated before the formation of the two-phase system. The emulsifier is either added to the coherent external phase, and the disperse phase is incorporated gradually, or the emulsifier is incorporated into the disperse internal phase.

Alternatively, the emulsifier may not be incorporated until after the two-phase system has been formed.

Examples of suitable emulsifiers include sodium dodecyl sulphate, lecithin, cetyl alcohol, cetylstearyl alcohol, sorbitan fatty acid esters, polyoxyethylene-sorbitan fatty acid esters, polyoxyethylene-fatty acid glycerides and polyoxyethylene-fatty acid esters.

In order to achieve as high as possible an active substance flux through the skin during the administration of the lerisetron TTS of the invention it has also proven to be particularly favourable for the active substance reservoir further to comprise at least one skin penetration enhancer. Examples of suitable skin penetration enhancers are substances selected from the group consisting of polyoxyethylene-fatty acid esters, polyoxyethylene-sorbitan fatty acid esters, sorbitan fatty acid esters, fatty acids, fatty alcohols, esters of fatty acids with methanol, ethanol or isopropanol, and esters of fatty alcohols with acetic acid or lactic acid. Examples of penetration enhancers are decanol, dodecanol, oleic acid, oleic acid diethanolamine, myristic acid, sorbitan monolaurate and polyoxylauryl ethers (e.g. Brij®). Preferred skin penetration enhancers used are polyoxyethylene-fatty alcohol ethers, with particular preference polyoxylauryl ethers (such as Brij®, e.g. Brij® 30). In order to optimize the active substance flux, it is also possible in accordance with the invention to use combinations of two or more penetration-enhancing substances.

The polymer matrix layer(s) of the active substance reservoir may further comprise plasticiz rs in ord r to influ nce the physical properties of th pressure-sensitiv adhesive matrix. Particularly suitable plasticizers are substances selected from the group consisting of hydrocarbons, alcohols, carboxylic acids and their derivatives, ethers, esters and amines. The concentration of the plasticizer(s), based on the active substance reservoir, may be from 0 to 30% by weight, it is preferably from 5 to 20% by weight.

In the simplest embodiment, the active substance reservoir of the lerisetron pharmaceutical preparations of the invention in TTS form is a single-layer polymer matrix. However, also envisaged in accordance with the invention are further embodiments which are distinguished by the TTS having a layer-form construction of the active substance reservoir, comprising at least two polymer matrix layers. In the case of such a multilayer active substance reservoir, it is possible to utilize the possibility for the individual polymer matrix layers to have different concentrations of active substance, skin penetration enhancer(s) or emulsifier(s).

Where a multilayer polymer matrix is used, there are also additional possibilities for variation in respect of the selection of the pressure-sensitive adhesive polymers. For example, it may be an advantage for at least two polymer matrix layers to differ in respect of the polymers involved in their construction, at least one polymer matrix layer preferably comprising polymer constituents from the group of polymers specified in claim 2. The individual layers may also have different silicone pressure-sensitive adhesives.

Where it is necessary to control the release of active substance and this control is not effected by other m chanisms, the delivery side (skin side) of the active substance r servoir may also be provid d with a control membrane which controls the delivery of the active substance to the skin. Membrane materials suitable for this purpose are known to the person skilled in the art.

In accordance with one particular embodiment, the active substance lerisetron may also be present in a pouchlike active substance reservoir in the preparation of the invention in the form of a TTS. This pouchlike reservoir is filled with a liquid, highly viscous, semisolid or thixotropic active substance matrix, it being particularly advantageous for the semisolid or thixotropic active substance reservoir to comprise a gel former. In this case, the reverse of the pouch, facing away from the skin, must be impermeable to active substance while the side facing the skin must be permeable to active substance. Optionally, a membrane which is permeable to active substance may also effect the control of the release of active substance.

In addition to the above-discussed active substance reservoir, the structure of the TTS of the invention comprises a backing layer, which is impermeable to active substance, and a removable protective film, which is likewise impermeable to active substance.

Suitable materials for the backing layer include a large number of skin-compatible polymer films, such as films of polyvinyl chloride, ethylene-vinyl acetate, vinyl acetate, polyethylene, polypropylene or cellulose derivatives, for example. Particularly suitable materials for the backing layer are polyesters which are distinguished by particular strength. In certain cases it may also be useful to provide the film material backing layer with an additional applied layer, for example by vapour deposition with m tals or other diffusion barrier additives such as silica, alumina or similar substances known to the person skilled in the art.

For the removable protective layer it is possible in principle to use the same materials as for the backing layer, subject to the proviso that this layer is subjected to an appropriate surface treatment, e.g. fluorosiliconization, so that it is removable from the pressure-sensitive adhesive layer it covers and can be removed prior to application of the TTS. As removable protective layers it is also possible, furthermore, to use other materials, such as polytetrafluoroethylene-printed paper, cellophane, polyvinyl chloride or the like, for example.

The lerisetron TTS of the invention are illustrated in more detail below with reference to a production example.

### EXAMPLE

The TTS of the invention may be produced as follows: first of all, the active substance lerisetron and an appropriate enhancer (e.g. Brij® 30) are dissolved in a solubilizer (e.g. 1,2-propanediol), the concentration of lerisetron approaching as far as possible the saturation solubility. If desired, the solution may also be supersaturated. This solution is introduced using a suitable stirring apparatus into the silicone adhesive, which is likewise in solution in a solvent, and is dispersed, so that a highly homogeneous liquid/liquid dispersion is formed.

This dispersion is coated homogeneously onto a backing film (e.g. backing layer) using an appropriate device. Subsequently, by means of controlled drying, the solvent of the silicone adhesive, and any fractions of the solubilizer, are removed. The laminate thus obtained is subsequently laminated with an additional film (e.g. protective film). Finally, individual TTS of a certain surface area are punched out and packaged in appropriate packaging. It has been found that with a TTS of this kind it is possible to achieve a delivery rate of lerisetron to the skin which is sufficient for therapeutic purposes. Measurements of the active substance permeation, made at 37° C. on human epidermis, gave active substance permeations of 100-600 μg/cr²d. This active substance flux is sufficient for therapeutic applications.

The example shows that the lerisetron TTS of the invention can be produced with production processes which permit simple and cost-effective production.

The lerisetron preparations of the invention in the form of TTS may be used advantageously for administering this active substance to patients transdermally for the purpose of the prevention and therapy of nausea and vomiting, in the case for example of nausea and vomiting induced as a consequence of chemotherapy or radiation therapy of the patient in question.

The transdermal administration proposed in accordance with the invention is particularly advantageous specifically in the situations mentioned above since it allows the active substance lerisetron to be administered systemically without passing through the gastrointestinal tract. Especially in the case of patients suffering from nausea and vomiting, reliable, safe and effective administration of medicaments by the oral route is almost impossible. Furthermore, the transdermal administration of the active substance lerisetron as proposed in accordance with the invention is also more patient-friendly than a corresponding oral administration, since in this way it is possible to avoid an unnecessary additional load on the already damaged or irritated gastrointestinal tract.

A patient suffering from nausea or vomiting, owing for example to radiation therapy or chemotherapy, may be treated with the active substance lerisetron to alleviate or eliminate these symptoms by sticking a lerisetron TTS of the invention onto the skin of the respective patient and repeating this operation, if necessary, at certain intervals of time. In this way it is possible to build up a systemic active-substance level which is therapeutically effective.

The lerisetron TTS of the invention are also suitable in particular for preventing nausea and or vomiting in respect of a forthcoming chemotherapy or radiation therapy. In this case, the lerisetron TTS are applied to the skin of the patient in question preferably before the beginning of the chemotherapy or radiation therapy.

In accordance with the invention the active substance lerisetron may therefore be used in order to produce a pharmaceutical preparation in the form of a TTS with the transdermal administration of the active substance lerisetron to human beings, this preparation being suitable for the prevention and therapy of nausea or vomiting, preferably for the prevention and therapy of vomiting or nausea induced by chemotherapy or radiation therapy.

1. Lerisetron pharmaceutical preparation in the form of a transdermal therapeutic system (TTS) which comprises a backing layer, connected to it an at least single-layer, pressure-sensitively adhesive, lerisetron active substance reservoir based on silicone pressure-sensitive adhesive(s), and a removable protective layer.

2. Lerisetron pharmaceutical preparation in the form of a transdermal therapeutic system which comprises a backing layer, connected to it an at least single-layer pressure-sensitively adhesive lerisetron active substance reservoir based on polymers from the group consisting of polyisobutylene, polyterpenes, ethylene-vinyl acetate copolymers, synthetic rubbers and hot-melt adhesives, and a removable protective layer.

3. Preparation according to claim 1 or 2, characterized in that the active substance reservoir comprises a combination of at least two pressure-sensitive adhesives selected from the group of pressure-sensitive adhesives specified in claim 1 and claim 2.

4. Preparation according to one of the preceding claims, characterized in that at least one matrix layer of the active substance reservoir comprises polymer constituents from the group of the substituted celluloses, preferably from the group of the methyl celluloses or ethyl celluloses.

5. Preparation according to one of the preceding claims, characterized in that the active substance concentration, bas d on the overall mass of the active substance layer(s), is in the range from 0.1 to 30% by weight, preferably in the range between 1 and 10% by weight, it being particularly preferred for the active substance concentration to match or exceed the saturation solubility.

6. Preparation according to one of the preceding claims, characterized in that the active substance lerisetron is present in molecularly disperse form in the active substance reservoir.

7. Preparation according to one of claims 1 to 5, characterized in that the active substance lerisetron is present in coarsely disperse form, in colloidal form, or as a suspension in the active substance reservoir.

8. Preparation according to one or more of the preceding claims, characterized in that the active substance reservoir comprises as a further constituent at least one solubilizer from the group of the polyhydric alcohols, preferably 1,2-propanediol.

9. Preparation according to one or more of the preceding claims, characterized in that the active substance reservoir comprises as a further constituent at least one skin penetration enhancer, preferably from the group of the polyoxyethylene-fatty alcohol ethers, with particular preference polyoxylauryl ethers.

10. Preparation according to claim 9, characterized in that the skin penetration enhancer(s) is (are) selected from the group consisting of polyoxyethylene-fatty acid esters, polyoxyethylene-sorbitan fatty acid esters, sorbitan fatty acid esters, fatty acids, fatty alcohols, esters of fatty acids with methanol, ethanol or isopropanol, and esters of fatty alcohols with acetic acid or lactic acid.

11. Preparation according to claim 9 or 10, characterized in that the active substance reservoir comprises at least two skin penetration enhancers in combination.

12. Preparation according to one or more of the preceding claims, characterized in that the active substance reservoir comprises as a further constituent at least one emulsifier, preferably from the group consisting of sodium dodecyl sulphate, lecithin, cetyl alcohol, cetylstearyl alcohol, sorbitan-fatty acid esters, polyoxyethylene-sorbitan fatty acid esters, polyoxyethylene-fatty acid glycerides and polyoxyethylene-fatty acid esters.

13. Preparation according to one or more of the preceding claims, characterized in that the active substance reservoir comprises as a further constituent at least one plasticizer, the concentration of the plasticizer(s), based on the active substance reservoir, being from 0 to 30% by weight, preferably from 5 to 20% by weight, and the plasticizer(s) preferably being selected from the group consisting of hydrocarbons, alcohols, carboxylic acids and their derivatives, ethers, esters and amines.

14. Preparation according to one or more of the preceding claims, characterized in that th transdermal th rapeutic system has a layer-form construction of the active substance reservoir, comprising at least two polymer matrix layers.

15. Preparation according to claim 14, characterized in that the at least two polymer matrix layers have different concentrations of active substance, skin penetration enhancer(s) or emulsifier(s).

16. Preparation according to claim 14 or 15, characterized in that the at least two polymer matrix layers differ in respect of the polymers involved in their construction, at least one polymer matrix layer preferably comprising polymer constituents from the group of polymers specified in claim 2.

17. Preparation according to one or more of the preceding claims, characterized in that the active substance reservoir of the TTS is pouchlike and is filled with a liquid, highly viscous, semisolid or thixotropic active substance matrix, the semisolid or thixotropic active substance reservoir preferably comprising a gel former.

18. Use of a preparation according to claims 1 to 17 for the transdermal administration of the active substance lerisetron for the prevention and therapy of nausea or vomiting in human beings.

19. Use of a preparation according to claim 18 for the prevention and therapy of vomiting or nausea induced by chemotherapy or radiation therapy in human beings.

20. Use of th active substance leris tron for producing a pharmaceutical preparation according to one of claims 1 to 17 for transdermal administration of th active substance lerisetron to human beings for the prevention and therapy of nausea or vomiting, preferably for the prevention and therapy of vomiting or nausea induced by chemotherapy or radiation therapy.

21. Method of administering the active substance lerisetron to a patient suffering from nausea or vomiting, especially vomiting or nausea induced by chemotherapy or radiation therapy, the said active substance being administered to the skin of the patient using a preparation according to one of claims 1 to 17.
